(19) United States
(12) Patent Application Publication
(10) Pub. No.: US 2011/0178545 A1
(43) Pub. Date: Jul. 21, 2011

(54) Nasal drip control devices

(76) Inventor: Isabel N. Gonzalez, McLean, VA (US)

(21) Appl. No.: 10/765,995

(22) Filed: Jan. 29, 2004

(51) Int. Cl. A61B 17/00

(52) U.S. Cl. 606/201

(57) ABSTRACT

The nasal drip cessation or control devices are devices which when strategically placed on the nose, causes the elimination or reduction of the nasal flow of mucus.

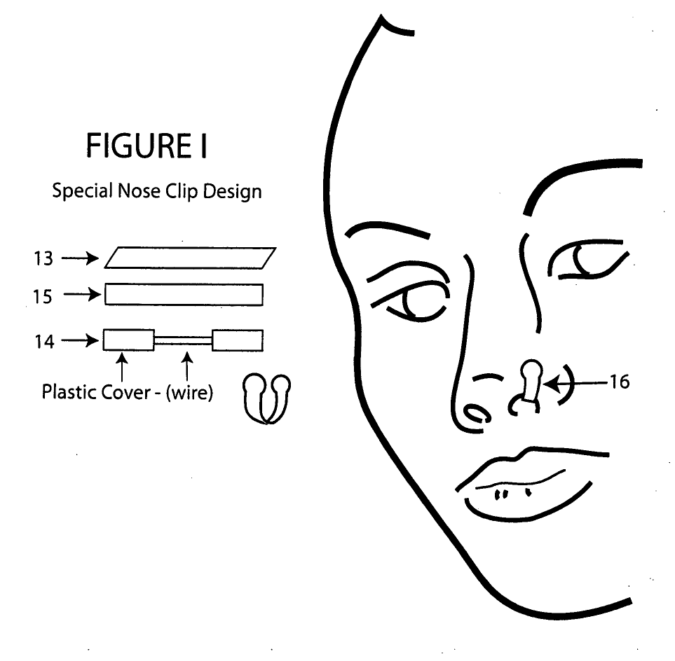

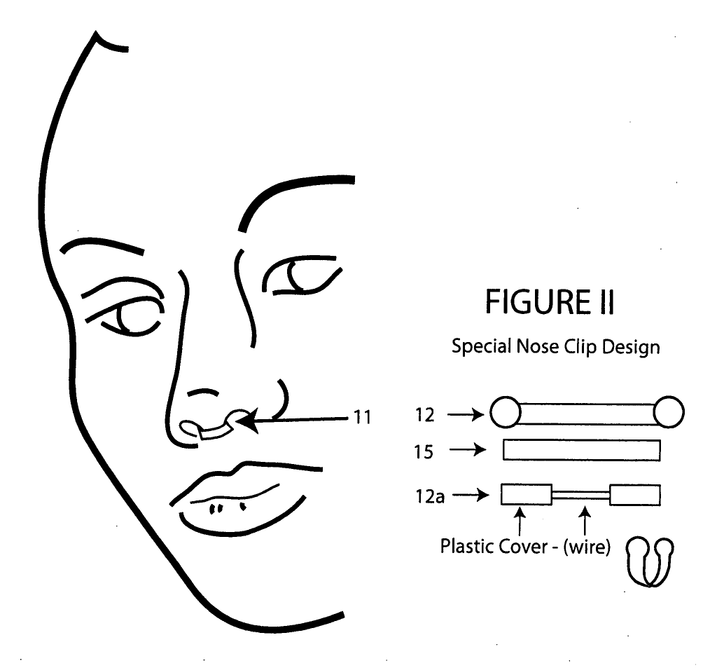

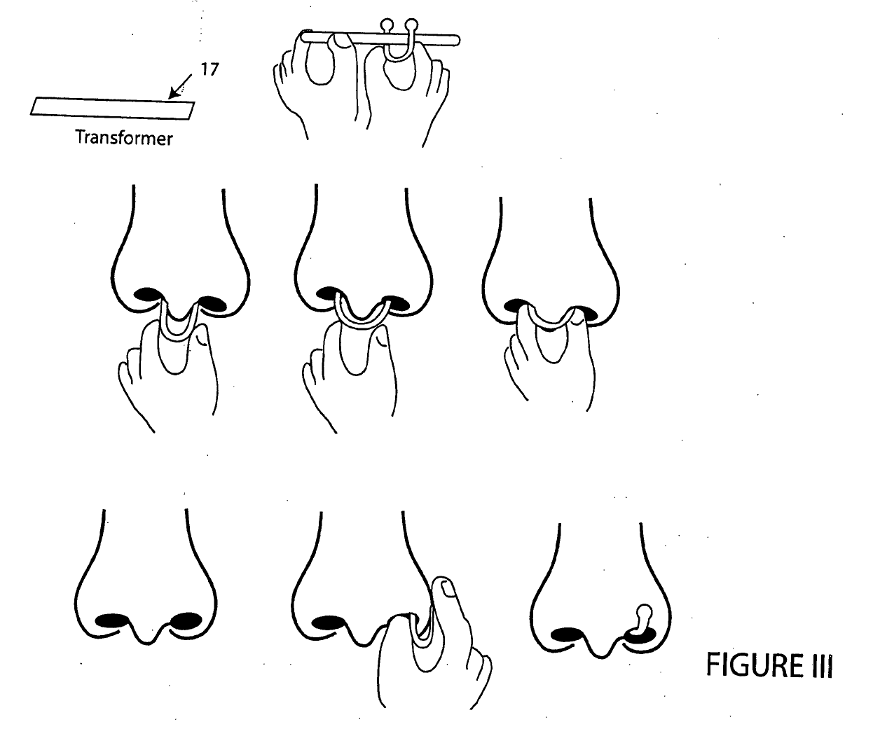

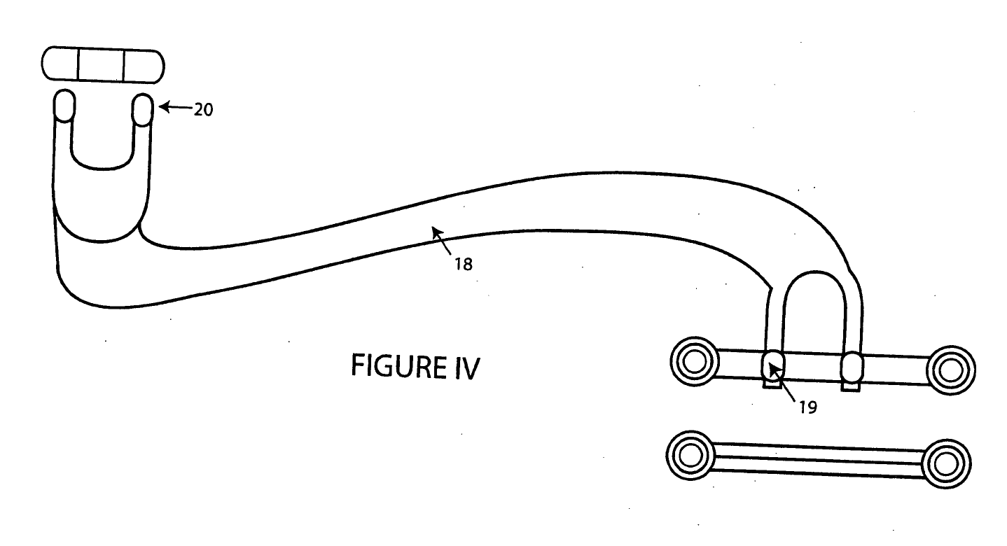

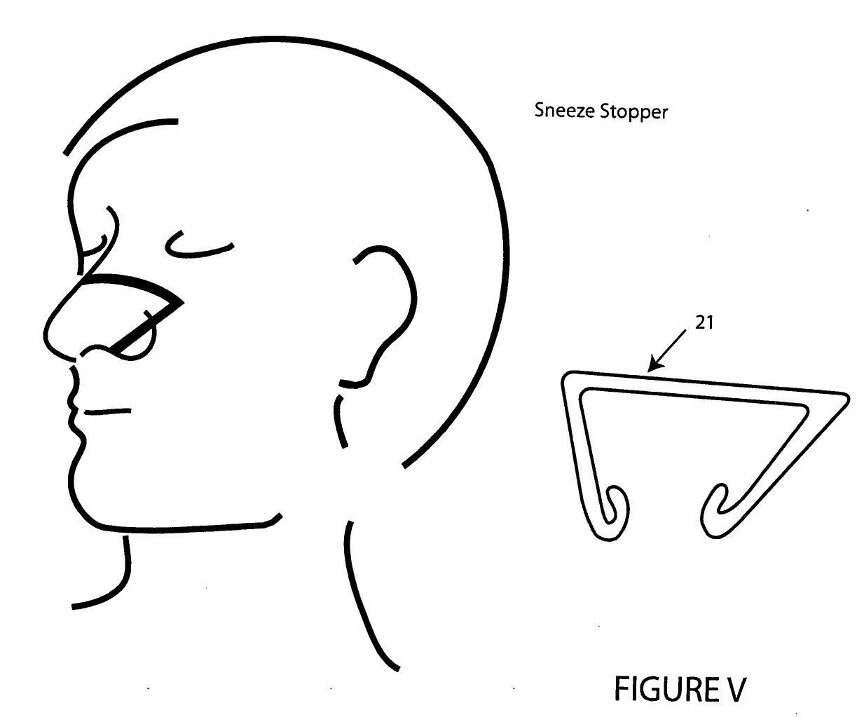

### FIELD OF INVENTION

The present invention relates to an apparatus for controlling nasal drip.

### BACKGROUND AND SUMMARY

Many people use the age-old trick of preventing a sneeze by placing a finger under the nose. This practice inspired the present inventor to search for a fixed device for this sneezing problem. In the process of searching for a solution to this problem, it was discovered that placing a device on certain pressure points on the nose not only stopped the sneezing but also the nasal and post-nasal drip.

### DESCRIPTION OF THE DRAWINGS

Figure I. Shows the nose strip on the nostril pressure point.

Figure II. Shows the nose strip on the front most membrane of the nose that divides the nostrils (nose middle loop).

Figure III. Shows the transformer and the placement of the device on the nose.

Figure IV. Shows a nose strip holder and placement device.

Figure V. Shows a sneeze stopper device and its placement on the nose.

### DETAILED DESCRIPTION OF THE INVENTION

The devices are a solution for the reduction and/or the elimination of the mucus flow and the irritants produced in the nose. These simply designed nasal devices, properly placed on the nose are a control measure for the runny nose, sneezing and post-nasal drip which the common colds, and most allergies cause.. These discomforts and irritabilities often result in more serious infections, such as, nose, ear, and throat infection, chest congestion, severe coughing, bronchial congestion, flu, pneumonia, bronchial asthma, chronic asthma, fatigue syndromes, and other respiratory problems.

It has been found that by strategically placing one or more of these nose devices on specific pressure points on the nose, the sneezing, runny nose flow, and post-nasal drip are eliminated.

The devices represent a novel method for the prevention of serious respiratory diseases.

Placement of the device (16) as shown in Fig. I, on the nostril pressure point, stops the sneezing and opens the nasal passages. These devices are adhesive strips (15), which are about 1/2 inch in length.

Fig. II shows the nose strip (11) on the nose middle loop (one pressure point). This strip placement diminishes the drip and prevents post-nasal drip. It is made of a metal wire (12a) coated with plastic (13). This strip is about an inch long.

These nose strips could have plastic balls on the ends to help hold the strips in place.

Fig. IV shows a nose strip holder and placement device (18). The holes (19) are there for holding the strip by passing the strip through the hole. The device holding an adhesive strip is shown in (20).

Fig. V shows a sneeze stopper device and its placement on the nose. This device has curves end or hooks that touch the pressure points of the nostril and holds the hooks in place on the nostrils.

I claim:

1. Nasal drip control devices which when placed on the pressure points of the nose, control or cause the cessation of the nasal production of mucus, comprising a plastic coated wire or an adhesive coated plastic strip.

2. The nasal drip control device of claim 1, which has plastic balls on the ends.

3. The nasal devices of claim 1 are a half-inch to an inch in length.

4. Nasal drip control device holder as shown in Figure IV to be held in the hand.

5. A sneeze stopper device that fits over the nose and has curved ends which fit on the nasal pressure points.
